(19) United States
(12) Patent Application Publication
(10) Pub. No.: US 2026/0112466 A1
(43) Pub. Date: Apr. 23, 2026

(54) METHODS AND SYSTEMS TO IDENTIFY EXPOSURE TO ENVIRONMENTAL TOXINS AND RISK ASSOCIATED WITH SUCH EXPOSURE

(71) Applicant: The Regents of the University of California, Oakland, CA (US)

(72) Inventors: Allan Balmain, Oakland, CA (US); Hang Chang, Oakland, CA (US); Jian-Hua Mao, Oakland, CA (US)

(21) Appl. No.: 19/364,880

(22) Filed: Oct. 21, 2025

Related U.S. Application Data
(60) Provisional application No. 63/710,953, filed on Oct. 23, 2024.

(51) Int. Cl. G16H 10/40; G01N 1/30; G06T 7/00; G06V 10/766; G06V 10/774; G06V 20/69

(52) U.S. Cl. CPC G16H 10/40; G01N 1/30; G06T 7/0014; G06V 10/766; G06V 10/774; G06V 20/695; G06V 20/698; G06T 2207/10056; G06T 2207/20081; G06T 2207/30024; G06V 2201/03

(57) ABSTRACT

Provided herein included methods and systems to identify exposure to environmental carcinogens, compounds, and other toxins are disclosed. Additionally provided, include methods and systems to identify risk associated with such environmental carcinogens, compounds, and other toxins. Various embodiments include machine learning models to identify cellular morphometric biomarkers (CMBs). Various methods and systems described herein are capable of using CMBs for diagnostic, prognostic, or forensic purposes. Some embodiments are directed to training a machine learning model for such using CMBs for diagnostic, prognostic, or forensic purposes, including (but not limited to) generating images of biological responses that can be used as a training data set. Additional embodiments are directed to computing devices and/or software that are capable of performing methods as described herein.

### CROSS-REFERENCE

This application claims the benefit of U.S. Provisional Patent Application No. 63/710,953 filed Oct. 23, 2024, which application is incorporated herein by reference in its entirety.

### STATEMENT REGARDING FEDERALLY SPONSORED RESEARCH

This invention was made with government support under CA278665 and R35 CA210018, awarded by the National Institutes of Health. The government has certain rights in the invention.

### I. INTRODUCTION

Exposure to certain environmental toxins can lead to biological responses. These biological responses include cellular response, molecular responses, etc. and may include malignancies. For example, exposure to some environmental toxins may be carcinogenic, in that they cause or increase the risk of developing a cancer. In some instances, environmental toxins may increase risk for aggressive and/or pernicious cancers. Current methods to understand the underlying cellular responses rely on-omic type analyses (e.g., genomics, transcriptomics, proteomics, etc.), including nucleic acid sequencing to identify underlying genetic variants and/or gene expression. These methods are financially burdensome and may require weeks or months of sample processing and analysis. Thus, there is a need for methods and systems that can identify exposure to environmental carcinogens (and other toxins) in a faster and more cost effective manner, and such is provided herein.

### II. SUMMARY

Machine learning and Artificial Intelligence (AI) algorithms can be used for assessment of patient prognosis based on high resolution scanning of tumor sections. Some studies have explored the possibility of using AI to distinguish between tumor classes and molecular subtypes. However, analysis of normal or tumor tissues after exposure to defined classes of environmental carcinogens, including mutagens and tumor promoters, other tissue damaging agents, or new drug treatments, has not been carried out. This approach provides important information on the effects of a wide variety of environmental factors linked not only to cancer, but to a host of other diseases including those associated with inflammation and ageing, such as Alzheimer's disease or metabolic conditions including diabetes. While many dietary or environmental factors have been linked to these conditions, the data are based on epidemiological associations, and the true causal factors, and their mechanisms of action, have not been identified.

Provided are methods for detecting the effects of different perturbations (e.g., environmental mutagens, tumor promoters, radiation, or other agents that induce toxicity or inflammation), on tissues and on tumors. In the working examples below, histological sections from normal and tumor tissues that were exposed to a wide variety of environmental agents, some of which play causal roles in tumor development or inflammation, were analyzed using an AI algorithm to detect Cellular Morphometric Biomarkers (CMBs). A CMB “score” based on combinations of CMBs associated with a particular perturbation (e.g., exposure to a chemical or radiation), can then be used to determine whether other tissues have been exposed to the same perturbation. This can be carried out for both normal tissue and tumors, giving insights into the individual history of exposures for a patient sample (e.g., a human patient).

Provided herein are methods and systems to identify exposure to perturbations (e.g., environmental carcinogens, radiation, compounds, and other toxins). Additionally provided, include methods and systems to identify risk associated with such environmental carcinogens, compounds, and other toxins. Various embodiments include machine learning models to identify cellular morphometric biomarkers (CMBs). Various methods and systems described herein are capable of using CMBs for diagnostic, prognostic, or forensic purposes. Some embodiments are directed to training a machine learning model for such using CMBs for diagnostic, prognostic, or forensic purposes. In some cases, a subject method includes generating images of biological responses that can be used as a training data set. Additional embodiments are directed to computing devices and/or software that are capable of performing methods as described herein.

### III. BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1A-1D. The development and double-blind validation of the CMB radiation scores. (A) overall study design of the CMB radiation scores development, validation and human-translation; (B) The CMB radiation scores in training cohort; (C) The CMB radiation scores on skin tissues across time points in double-blind validation cohort; and (D) The CMB radiation scores on mammary glands across time points in double-blind validation cohort. The p-values were obtained from the Mann-Whitney test.

FIG. 2A-2B. The CMB radiation scores in chemically induced tumors and treated normal tissues. (A) Comparison of the CMB radiation scores between spontaneous liver tumors and chemically induced liver tumors. (B) Comparison of the CMB radiation scores between control normal skin and DMBA-TPA treated skin. The p-values were obtained from the Mann-Whitney test.

FIG. 3A-3F. Human pan-cancer translational study. (A-D) the association between the CMB radiation scores and aneuploidy score, fraction of genome altered, mutation counts and burden, respectively, in human pan-cancer; (E-F) the association between the CMB radiation scores and progression-free and overall survival, respectively, in human pan-cancer.

FIG. 4. CMB-BP-Network, the association between CMBs and biological process (BP) in human pan-cancer.

FIG. 5A-5C. Radiation associated CMBs. (A) Representative example of each radiation associated CMB and its local neighborhood (1200 pixel×1200 pixel; 133.2 μm×133.2 μm; pixel size: 0.111 μm×0.111 μm); (B) Abundance of each radiation associated CMB in Sham and 10 cGy treatment groups (The p-values were obtained from Mann-Whitney test); and (C) CMB-RELS model.

FIG. 6A-6B. Comparison of CMB-RELS between gamma- and ⁵⁶Fe-ions-irradiated samples. (A) Skin samples. (B) Mammary tissue samples. The p-values were obtained from Mann-Whitney test.

FIG. 7A-7B. Comparison of CMB-RELS between sham-treated and irradiated samples. (A) Skin samples. (B) Mammary tissue samples. The p-values were obtained from Mann-Whitney test.

FIG. 8A-8K. Radiation associated CMBs accurately predict radiation status. Classification performance during cross-validation on hold-out samples in training cohort (A, E); classification performance on all samples (B, F), skin tissues (C, G), and mammary glands (D, H) in the double-blind validation cohort; and classification performance over time points on all samples (I), skin tissues (J), and mammary glands (K) in the double-blind validation cohort. The p values were obtained from the Kruskal-Wallis test.

FIG. 9. Association of CMB-RELS with aneuploidy score, fraction genome altered, mutation counts and burden in each cancer type from TCGA pan-cancer cohort. The p values were obtained from the Kruskal-Wallis test.

FIG. 10. Association of CMBs with tumor microenvironments (TMEs) from TCGA pan-cancer cohort. The p values were obtained from the spearman correlation analysis (*: p<0.05; **: p<0.01; ***: p<0.001).

FIG. 11. Mice were treated with the mutagens methylnitrosourea (MNU) or dimethylbenzanthracene (DMBA), or with a strong promoter (Tetradecanoyl-phorbol-acetate, TPA) or a weak promoter (Retinoyl-phorbol acetate RPA). AI-analyzed scans of histological sections of the skin showed minimal overlap between these different treatments based on 10 samples for each category.

FIG. 12. Mice were treated (at National Toxicology Program, NIEHS) chronically for 2 years with the series of chemicals shown, all of which are known or suspected of causing cancer in humans. Only 2 of these chemicals showed clear signs of being mutagenic as shown by tumor genome sequencing. A control group developed spontaneous tumors which have a longer latency. Analysis of induced and spontaneous tumor histology sections showed that CMBs for most chemicals fell into distinct groups, although with some overlap, in spite of the fact that some groups had only 4 animals.

FIG. 13. Survival of human patients with hepatocellular carcinoma is correlated with a CMB score derived from mouse liver tumors induced by exposure to any of the chemicals shown in FIG. 13. Patient tumors with no significant score are classified as “spontaneous.”

FIG. 14. Data demonstrating that cellular morphometric biomarkers (CMBs) can distinguish between different chemical exposures.

FIG. 15. Data demonstrating replication of CMB profiles using independent samples.

FIG. 16. Data demonstrating validation of selected CMBs derived from an independent set of samples of skin squamous carcinomas and matched normal skin samples.

### IV. DEFINITIONS

The terms “subject,” “individual,” “patient,” and “participant” are used interchangeably herein and refer to an individual organism, e.g., a mammal, including, but not limited to, canines, felines, porcines, bovines, equines, humans, non-human primates, murines, ovines, caprines, ungulates, simians, mammalian farm animals, mammalian sport animals, and mammalian pets. In some cases, an “individual” is a human.

By “small molecule” compound is meant a compound having a molecular weight of 1000 atomic mass units (amu) or less. In some embodiments, the small molecule is 900 amu or less, 750 amu or less, 500 amu or less, 400 amu or less, 300 amu or less, or 200 amu or less. In some instances, the small molecule is not made of repeating molecular units such as are present in a polymer.

The term “antibody” may include an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the cell surface molecule of the target cell, including, but not limited to single chain Fv (scFv), Fab, (Fab′)2, (scFv′)2, and diabodies; chimeric antibodies; monoclonal antibodies, human antibodies, humanized antibodies (e.g., humanized whole antibodies, humanized half antibodies, or humanized antibody fragments, e.g., humanized scFv); and fusion proteins comprising an antigen-binding portion of an antibody and a non-antibody protein. According to some embodiments, the antibody is selected from an IgG, Fv, single chain antibody, scFv, Fab, F(ab′)2, or Fab′. In certain embodiments, the antibody is a nanobody (an antibody fragment consisting of a single monomeric variable antibody domain—also known as a single-domain antibody (sdAb)), a monobody (a synthetic binding protein constructed using a fibronectin type III domain (FN3) as a molecular scaffold), or a Bi-specific T-cell engager (BiTE).

As used herein, a “therapeutic agent” is a physiologically or pharmacologically active substance that can produce a desired biological effect in a targeted site in an animal, such as a mammal or in a human. The therapeutic agent may be any inorganic or organic compound. A therapeutic agent may decrease, suppress, attenuate, diminish, arrest, or stabilize the development or progression of disease, disorder, or cell growth in an animal such as a mammal or human. Examples include, without limitation, peptides, proteins, nucleic acids (including siRNA, miRNA and DNA), polymers, and small molecules. In various embodiments, the therapeutic agents may be characterized or uncharacterized.

By “treat” or “treatment” is meant at least an amelioration of the symptoms associated with the disease state of the subject, where amelioration is used in a broad sense to refer to at least a reduction in the magnitude of a parameter, e.g. symptom, associated with the disease state being treated. As such, treatment also includes situations where the disease state, or at least symptoms associated therewith, are completely inhibited, e.g. prevented from happening, or stopped, e.g. terminated, such that the subject no longer suffers from the disease state, or at least the symptoms that characterize the disease state.

A “sample” in the context of the present disclosure refers to any biological sample that is isolated from a subject. A sample can include, without limitation, an aliquot of body fluid, whole blood, PBMC (white blood cells or leucocytes), tissue biopsies, synovial fluid, lymphatic fluid, ascites fluid, and interstitial or extracellular fluid. A sample may include any body tissue of interest and/or a fluid, such as, but not limited to, blood, sweat, saliva, and urine. A sample can include sections of tissues such as biopsy and frozen sections taken for histological purposes. In some cases, the sample may be a tissue biopsy, e.g., from kidney or gastrointestinal tract such as stomach, small intestine, or large intestine including but not limited to the cecum, colon, or rectum, spleen, other lymphoid tissues, liver, lung, pancreas, breast, bone, prostate, cervix, testes, ovaries, tonsil, or other organ, and/or cells derived therefrom.

“Blood sample” can refer to whole blood or a fraction thereof, including blood cells, white blood cells or leucocytes. Samples can be obtained from a subject by means including but not limited to venipuncture, biopsy, needle aspirate, lavage, scraping, surgical incision, or intervention or other means known in the art.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. As such, the articles “a” and “an” are used herein to refer to one or to more than one (i.e., to at least one) of the grammatical object of the article. By way of example, “an element” means one element or more than one element. Thus, for example, reference to “a cell” includes a plurality of such cells and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. For example, it is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### V. DETAILED DESCRIPTION

Provided herein included methods and systems to identify exposure to a perturbation (e.g., environmental carcinogens, radiation, compounds, and other toxins) are disclosed. Additionally provided, include methods and systems to identify risk associated with such perturbations. Various embodiments include machine learning models to identify cellular morphometric biomarkers (CMBs). Various methods and systems described herein are capable of using CMBs for diagnostic, prognostic, or forensic purposes. Some embodiments are directed to training a machine learning model for such using CMBs for diagnostic, prognostic, or forensic purposes. In some cases, a subject method includes generating images (e.g., of training a machine learning model histological sections) of biological responses that can be used as a training data set. Additional embodiments are directed to computing devices and/or software that are capable of performing methods as described herein.

### Methods

The present disclosure provides methods for identifying exposure to a perturbation (e.g., environmental carcinogens, compounds, and other toxins) and risks associated with such exposure. Many embodiments utilize histological images of sample(s) obtained from a subject. In various instances, systems and methods herein are utilized to detect and/or assess risk of a subject that has been exposed to an environmental toxin. In some instances, the systems and methods herein are utilized to detect and/or assess risk of a subject that is suspected of having been exposed to an environmental toxin. In certain instances, the systems and methods herein are utilized as part of routine screening to identify exposure to an environmental toxin.

Such exposures may be indicative or causative of certain diseases or disorders, including inflammation, inflammatory diseases, diseases associated with inflammation, and cancers. Some diseases associated with inflammation include (but are not limited to) autoimmune diseases (e.g., rheumatoid arthritis, lupous, multiple sclerosis, inflammatory bowel disease, psoriasis, ankylosing spondylitis, and type 1 diabetes), chronic inflammatory diseases (e.g., atherosclerosis, asthma, chronic obstructive pulmonary disease, Alzheimer's disease, and obesity-related inflammation), allergic or hypersensitivity disorders (e.g., eczema, hay fever, and contact dermatitis), and other diseases linked to inflammation (e.g., celiac disease, gout, sarcoidosis, chronic sinusitis, and fibromyalgia. The terms “cancer” and “cancerous” refer to or describe the physiological condition in mammals that is typically characterized by unregulated cell growth/proliferation. Examples of cancers include but are not limited to: solid tumors and liquid tumors, carcinoma, lymphoma, blastoma, sarcoma, myeloma, leukemia, or any combination thereof. Examples of such cancers include renal cancer; kidney cancer; glioblastoma multiforme; metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and ciliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgendependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; and bladder cancers such as but not limited to transitional cell carcinoma, squamous cell cancer, adenocarcinoma, carcinosarcoma. In some embodiments, the cancer is myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, or papillary adenocarcinomas.

Some methods and systems herein may be used to screen compounds for physiological effect (e.g., treatment). In some instances, methods and systems herein can be used to identify medical compounds that are effective in treating, ameliorating, and/or alleviating an underlying medical condition—e.g., cancer, inflammatory condition, etc.—from which an individual or subject suffers. In various instances, an individual

In some instances, embodiments are directed to generating a reference library of cellular morphometric biomarkers (CMBs). In some instances, such a library may be constructed using histological images of tissue. In some embodiments, the histological images comprise a biopsy of tissue from an organism (e.g., murine, human, bovine, etc., as described herein). In some instances, the tissue has been treated with a perturbation to induce a biological response. A perturbation in accordance with many embodiments is a chemical compound or radiation source (e.g., from radioactive decay and/or ionizing radiation). Additionally or alternatively, such histological images may be used as training data for a machine learning model-such as machine learning models that can be used for disease diagnosis, prognosis, and/or forensics.

In many instances, histological images may be generated de novo by intentionally exposing tissue (e.g., normal tissue) to one or more perturbations. In some cases, this exposure is in vivo, e.g., mice can be intentionally exposed to a perturbation, and tissue samples can then be collected from exposed and unexposed mice to generate histological images. In this context, “normal tissue” refers to physiologically typical tissue that is growing in a customary or expected growth pattern for the particular tissue type and species and is not exhibiting a diseased growth pattern, a mutated growth pattern, and/or any other abnormal or atypical growth pattern. In some cases, the unexposed tissue (e.g., reference tissue) is normal tissue. In some cases, the unexposed tissue (e.g., reference tissue) is not normal tissue, but nonetheless serves as a control for the exposed tissue. As an illustrative example, in some cases, the exposed and unexposed tissue both harbor a mutation, e.g., a cancer causing mutation, and the analysis is therefore based on exposure of that type of tissue to a perturbation (e.g., chemical compound, radiation, and the like). In various instances, the perturbation is a chemical compound and/or radiation source. In some instances, a perturbation may include more than one compound or radiation—e.g., exposing tissue to both a chemical compound and a radiation source.

Such chemical compounds may be a mutagen, a tumor promoter, a tumor anti-promoter, an anti-inflammatory, a chemotherapeutic, an immunotherapeutic, and/or any other compounds with a known or unknown ability to induce a biological response. Such compounds may be applied to the tissue via an appropriate mechanism, including topical exposure or internal exposure (e.g., via injection or puncture).

Radiation may be selected from any type of radiation, including ionizing radiation (e.g., alpha-particles, beta-particles, gamma-radiation, x-rays, neutron radiation) or non-ionizing radiation (e.g., ultraviolet (UV) radiation, visible light, infrared (IR) radiation, microwaves, radio waves, low frequency waves). Radiation sources may be selected from any appropriate source, such as a light source (e.g., light bulb), antenna, etc. Ionizing radiation sources may be selected from a nuclide, such as (but NOT Limited to) ²³⁵U, ²³⁸U, ²³⁹Pu, ²³⁸Pu, ²³²Th, ²²⁶Ra, ¹⁴C, ¹³¹I, ¹³⁷Cs, ⁸⁵Kr, ⁵⁶Fe, ⁹⁰Sr, ²¹⁰Po, ⁹⁹Tc, ²²²Rn, any Other Radiation Source, and Combinations Thereof.

In various instances, the tissue type may be in vivo (e.g., as part of an entire organism) or in vitro (e.g., cultured cells, cultured tissue, isolated tissue, isolated cells, etc.) In some instances, the tissue comprises an organoid, such as a mouse organoid and/or human organoid. In various instances, a piece of in vivo skin tissue (e.g., dermis) is exposed to the perturbation. In various instances, the in vivo skin tissue is part of a murine species (e.g., mouse). In some instances, the individuals are isogenic, such as through inbreeding, cloning, and/or any other method that reduces genetic heterogeneity. In some instances, the individuals are heterogeneous in order to capture genetic components to biological responses to perturbations. In some instances, the individuals are genetically modified (e.g., knock-in, knock-out, transgenic, cisgenic, mutated, etc.) in situations where particular pathways may be under investigation or otherwise of interest. In some embodiments the tissue is genetically modified to induce a phenotype selected from: genome instability, membrane perturbation, increased inflammation, tumor susceptibility, and combinations thereof.

In many instances, multiple individuals of a murine species are exposed to a perturbation, where each individual is exposed to a different perturbation (e.g., a first individual is exposed to a chemical compound, while a second individual is exposed to radiation, etc.) and/or a different dose of a perturbation (e.g., a first individual is exposed to a low dose of a chemical compound, while a second individual is exposed to a high dose of the same chemical compound, etc.). In some instances, an individual is exposed acutely (e.g., a single dose), while some tissue is exposed chronically (e.g., multiple exposures over a series of time). Such chronic exposure can include a longer exposure time (e.g., exposure for 24 hours, 36 hours, 48 hours, 60 hours, 72 hours, or greater) or multiple exposures over a period of time (e.g., 2 doses, 3 doses, 4 doses, 5 doses, 10 doses, 15 doses, 20 doses, 25 doses, or greater. During chronic exposure, each individual dose may be the same—e.g., all doses have the same dosage amount. In some instances, each dose in chronic exposure may vary in dosage—for example, an initial dose may have a first dosage, while subsequent doses may gradually increase (or decrease) over the course of chronic exposure. It should be noted that the doses may alter in any other pattern, including random over the course of chronic exposure. As noted previously, an individual or tissue can be exposed to multiple perturbations, which can also be applied to acute and chronic exposure—e.g., an acute exposure to a first compound combined with chronic exposure of a second compound.

In some instances, at least one individual is used as a reference sample or reference tissue, in that it is not exposed to a perturbation.

Histological slides may be prepared from the test (e.g., exposed to a perturbation) and reference tissue in accordance with any acceptable means, including biopsy, excision, vivisection, and/or any other form of tissue removal. Such tissue removal may be performed pre- and/or or post-mortem. After excision or removal, such tissue may be stained for imaging or visualization. Many such stains are known in the art, including hematoxylin and eosin, toluidine blue, methylene blue, periodic acid-Schiff, Masson's trichome, Gomori's trichome, Van Gieson stain, Verhoeff-Van Gieson, Sirius Red, Reticulin stain, Weigert's stain, alcian blue, mucicarmine stain, oil red O, Sudan Black B, Gram stain, Ziehl-Neelsen stain, Giemsa stain, luxol fast blue, Nissl stain, Bielschowsky stain, Feulgen stain, Mayer's hematoxylin. Some preferred embodiment elect hematoxylin and eosin, because hematoxylin stains nuclei blue/purple, and eosin stains cytoplasm and extracellular matrix pink.

In some instances, a portion of the tissue may be used for additional genetic, chemical, and/or biochemical analysis. Exemplary proteomic analyses include (but are not limited to) identifying proteins, allozymes, isozymes, protein isoforms, protein modification (e.g., post-translational modifications), protein/peptide sequences, etc. Exemplary genomic analyses include cytogenetic analyses (sequencing based or karyotypically), genomic sequencing, bisulfite sequencing (e.g., to identify DNA methylation), RNA sequencing (bulk and/or single cell), and/or any other relevant form of analysis. With such-omic data, some embodiments may identify underlying genetic, genomic, biochemical, and/or other biological phenomena that affect the or characterize the cancer, tumor, or other disease or condition.

In some cases, genome-wide gene expression data can be correlated with CMB perturbation scores, and thus CMB perturbation scores can be used to predict underlying gene expression changes in a given sample, thus shedding light on the biological mechanisms related to a given perturbation.

Imaging the tissue may occur through any applicable method or system, including via a microscope, slide scanning microscope, pathology scanner, etc. The histological images may be saved on any appropriate media (e.g., digital or analog), including digital memory, such as volatile and non-volatile memory types. Details about the sample, such as type of perturbation, exposure type (e.g., chronic/acute/etc.), dosing regimen, dosage amounts, etc., may be stored as metadata along with the images.

The images along with any associated metadata may be provided to a machine learning model, such as described herein. The machine learning model may identify a morphometric profile comprised of a set of CMBs to differentiate each tissue type and treatment.

Many embodiments are directed to machine learning models and methods of training machine learning models that are trained to identify one or more CMBs within a subject sample. In some embodiments, the machine learning model identifies a set of CMBs from the images (e.g., histological images) of the reference and test tissues based on the morphometric features. Reference tissues are comparable to the test tissues, but unlike the test tissue, have not be intentionally exposed to a give perturbation. The CMBs are based on identifying morphometric features within the histological images. CMBs can be used for identifying significant morphometric features that can be used to score severity and/or differentiate perturbations. Such machine learning models may comprise a neural network, such as an artificial neural network, a convolutional neural network, a deep learning neural network, and/or other applicable model. For models such as these, and for more information related CMBs and examples of how CMBs can be used, see, e.g., US 2024/0047007 and Liu, X.-P., et al., “Clinical significance and molecular annotation of cellular morphometric subtypes in lower-grade gliomas discovered by machine learning,” Neuro-Oncology, 25(1):68-81 (2023); the disclosures of which are hereby incorporated by reference in their entireties.

In various embodiments, the machine learning model comprises a network layer comprising a set of dictionary elements, such that the dictionary elements each represent a morphometric feature. To identify the CMBs, various embodiments utilize stacked predictive sparse decomposition (SPSD) to identify a collection of dictionary elements. Such dictionary elements may be identified via supervised learning, unsupervised learning, or semi-supervised learning. In some preferred embodiments, the SPSD uses unsupervised learning to identify the collection of dictionary elements. Additional details regarding SPSD may be found in Chang, H., et al., “Stacked Predictive Sparse Decomposition for Classification of Histology Sections,” Int J Comput Vis. 113(1):3-18(2015); the disclosure of which is incorporated by reference in its entirety.

In some embodiments, SPSD uses spatial pyramid matching of the collection of dictionary elements to generate a set of dictionary elements to be used in a network layer of the machine learning model. In various embodiments, the network layer comprises at least 4 dictionary elements, at least 8 dictionary elements, at least 16 dictionary elements, at least 32 dictionary elements, at least 64 dictionary elements, at least 128 dictionary elements, at least 256 dictionary elements, at least 512 dictionary elements, at least 1024 dictionary elements, or at least 2048 dictionary elements, where the upper limit of such dictionary elements is a maximum based on storage capacity and/or computing power of a computing device. In some embodiments, the network layer comprises approximately 4 dictionary elements, approximately 8 dictionary elements, approximately 16 dictionary elements, approximately 32 dictionary elements, approximately 64 dictionary elements, approximately 128 dictionary elements, approximately 256 dictionary elements, approximately 512 dictionary elements, approximately 1024 dictionary elements, or approximately 2048 dictionary elements. In some embodiments, the network layer comprises 4 or fewer dictionary elements, 8 or fewer dictionary elements, 16 or fewer dictionary elements, 32 or fewer dictionary elements, 64 or fewer dictionary elements, 128 or fewer dictionary elements, 256 or fewer dictionary elements, 512 or fewer dictionary elements, 1024 or fewer dictionary elements, or 2048 or fewer dictionary elements.

Training of such machine learning models may begin with providing a set of histological images, such as images described previously. In various embodiments, the machine learning model comprises a network layer comprising a set of dictionary elements, such that the dictionary elements each represent a morphometric feature. In some embodiments, the machine learning model identifies a set of CMBs from the images of the reference and test tissues based on the morphometric features.

After training, some embodiments generate a set of CMBs from 1 to the number of CMBs in the network layer. For example, the set of CMBs comprises at least 1 CMBs, at least 2 CMBs, at least 3 CMBs, at least 4 CMBs, at least 5 CMBs, at least 10 CMBs, at least 15 CMBs, at least 20 CMBs, at least 25 CMBs, at least 30 CMBs, at least 35 CMBs, at least 40 CMBs, at least 45 CMBs, at least 50 CMBs, at least 75 CMBs, at least 100 CMBs, at least 125 CMBs, at least 150 CMBs, at least 200 CMBs, or at least 250 CMBs. In certain embodiments, the set of CMBs comprises approximately 1 CMBs, approximately 2 CMBs, approximately 3 CMBs, approximately 4 CMBs, approximately 5 CMBs, approximately 10 CMBs, approximately 15 CMBs, approximately 20 CMBs, approximately 25 CMBs, approximately 30 CMBs, approximately 35 CMBs, approximately 40 CMBs, approximately 45 CMBs, approximately 50 CMBs, approximately 75 CMBs, approximately 100 CMBs, approximately 125 CMBs, approximately 150 CMBs, approximately 200 CMBs, or approximately 250 CMBs.

Once a set of CMBs have been identified via machine learning, additional embodiments identify a subset of these CMBs that significantly correlate to the test tissue images versus the reference tissue images. In such embodiments, the subset of significantly correlated CMBs comprises the CMBs with the largest variances in abundance contributing to 50% or more, 55% or more, 60% or more, 65% or more, 70% or more, 75% or more, 80% or more, 85% or more, 90% or more, or 95% or more of total data variation. In some preferred embodiments, the CMBs with the largest variances in abundance contributing to 95% or more of total data variation. In some embodiments, a method comprises identifying the CMBs with the largest variances in abundance contributing to 50% or more, 55% or more, 60% or more, 65% or more, 70% or more, 75% or more, 80% or more, 85% or more, 90% or more, or 95% or more of total data variation. In some preferred embodiments, a method comprises identifying the CMBs with the largest variances in abundance contributing to 95% or more of total data variation.

Following identification of CMBs with the largest variances in abundance contributing to total data variation, additional embodiments identify the CMBs that are predictive of test tissue (e.g., exposed to a perturbation) versus reference tissue. In some embodiments, the predictive CMBs are identified by step-wised multivariate linear regression.

In various embodiments, the subset of CMBs that significantly correlate with the images of the test tissue versus the images of the reference tissue further includes generating a CMB perturbation scoring algorithm based on the identified subset of significantly associated CMBs. In certain situations, the CMB perturbation scoring algorithm has the formula (Equation 1):

$$CMB perturbation score = Intercept + {\sum }_{i = 1}^{N} ( coefficient of {CMB}^{i} ) * ( {CMB}^{i} ) \qquad ( 1 )$$

Where “N” is the number of CMBs in the identified subset of significantly associated CMBs, and the coefficient of CMBⁱ is obtained for each significantly associated CMB using step-wised multivariate linear regression.

Further embodiments test or evaluate a trained machine learning model by its ability to reconstruct a cellular object based on at least one CMB. For example, the subset of significantly correlated CMBs may be used to reconstruct a cellular object. For example, given a set of CMBs, the machine learning model should be able to reconstruct the cellular features that the CMBs describe.

Additional embodiments are directed to using a machine learning (e.g., as trained in the prior sections) to output CMB values, a CMB perturbation score, a risk score, a diagnosis, a prognosis, and/or prediction of a biological response based on an inputted histological image. In many instances, a histological image from an individual (e.g., patient, subject, etc.) for evaluation. In some instances, the individual being evaluated is the same species as the training data for the machine learning model. In certain instances, the individual being evaluated is a different species than the training data for the machine learning model—for example, the machine learning model may have been trained using mouse tissue, while the individual being evaluated is human. It should be noted that these species are merely exemplary and other species or species combinations (e.g., mouse and dog, dog and human, dog and cat, etc.) are within the scope of the present disclosure.

In many such embodiments, the individual possesses an abnormal tissue growth which may be of concern. In some instances, the individual is suspected of having been exposed to a perturbation (e.g., chemical compound and/or radiation source). In some forensic instances, the perturbation is unknown.

A histological image from an individual may be obtained from a biopsy or other excision. The histological image may be input into a computing system (e.g., as described herein), which communicates with a trained machine learning model to determine one or more of the outputs noted previously. In the case of a CMB perturbation score, such as provided in Equation 1.

In some embodiments, the output is a diagnosis and/or prognosis. In some instances, the diagnosis and/or prognosis are cancer-related (i.e., the diagnosis is a cancer-related diagnosis and/or the prognosis is a cancer-related prognosis). A diagnosis and/or prognosis may be based on a CMB perturbation score, such as described previously.

Additionally or alternatively, some embodiments provide an exposure risk for exposure to a harmful compound. Such methods may be used to identify one or more environmental toxins that affect the health of the individual. Such exposure risk may be based on a CMB perturbation score. In some instances, the exposure risk indicates a risk of developing a cancer. In certain embodiments, the exposure risk indicates a record of past exposure to a compound. In some instances, the model outputs the particular perturbation (e.g., compound or class of compounds, radiation source) to which the individual has been exposed.

In further embodiments, a method or system as detailed herein may output or determine a method to mediate or remediate the perturbation or ailment. For example, a mediation method may include a medical intervention or treatment, including (but not limited to) excision, chemotherapy, immunotherapy, radiotherapy, or any combination thereof. Such interventions may comprise small molecule treatments, antibody-based treatments, and/or other therapeutic agents for treatment.

In certain instances, the systems and/or methods output an environmental remediation method, such as selected from: replacing pipes, phytoremediation, topsoil replacement, increasing water purity, increasing air circulation, increasing air purity, sequestering or encapsulating lead paint, or any combination thereof.

### Systems and Computer Implemented Methods

Aspects of the invention additionally include systems configured to perform the above-described methods and systems to identify exposure to environmental carcinogens, compounds, and other toxins.

In some instances the systems further includes one or more computers for complete automation or partial automation of the methods described herein. In some embodiments, systems include a computer having a computer readable storage medium with a computer program stored thereon.

In embodiments, the system includes an input module, a processing module and an output module. The subject systems may include both hardware and software components, where the hardware components may take the form of one or more platforms, e.g., in the form of servers, such that the functional elements, i.e., those elements of the system that carry out specific tasks (such as managing input and output of information, processing information, etc.) of the system may be carried out by the execution of software applications on and across the one or more computer platforms represented of the system.

Systems may include a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, Python, other high-level or low-level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques. The processor may be any suitable analog or digital system. In some embodiments, the processor includes analog electronics which provide feedback control, such as for example negative feedback control.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described including a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid-state storage devices (including magnetic or optical disks or tape or RAM, or any other suitable device, either fixed or portable). The processor may include a general-purpose digital microprocessor suitably programmed from a computer readable medium carrying necessary program code. Programming can be provided remotely to processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (i.e., smartphone).

In some embodiments, systems according to the present disclosure may be configured to include a communication interface. In some embodiments, the communication interface includes a receiver and/or transmitter for communicating with a network and/or another device. The communication interface can be configured for wired or wireless communication, including, but not limited to, radio frequency (RF) communication (e.g., Radio-Frequency Identification (RFID), Zigbee communication protocols, WiFi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, an RS-232 port, or any other suitable electrical connection port to allow data communication between the subject systems and other external devices such as a computer terminal (for example, at a physician's office or in hospital environment) that is configured for similar complementary data communication.

In one embodiment, the communication interface is configured for infrared communication, Bluetooth® communication, or any other suitable wireless communication protocol to enable the subject systems to communicate with other devices such as computer terminals and/or networks, communication enabled mobile telephones, personal digital assistants, or any other communication devices which the user may use in conjunction.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or WiFi connection to the internet at a WiFi hotspot.

In one embodiment, the subject systems are configured to wirelessly communicate with a server device via the communication interface, e.g., using a common standard such as 802.11 or Bluetooth® RF protocol, or an IrDA infrared protocol. The server device may be another portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touch-screen.

In some embodiments, the communication interface is configured to automatically or semi-automatically communicate data stored in the subject systems, e.g., in an optional data storage unit, with a network or server device using one or more of the communication protocols and/or mechanisms described above.

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a workstation, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows, iOS, Oracle Solaris, Linux, IBM, Unix, and others.

Aspects of the present disclosure further include non-transitory computer readable storage mediums having instructions for practicing the subject methods. Computer readable storage mediums may be employed on one or more computers for complete automation or partial automation of a system for practicing methods described herein. In certain embodiments, instructions in accordance with the method described herein can be coded onto a computer-readable medium in the form of “programming”, where the term “computer readable medium” as used herein refers to any non-transitory storage medium that participates in providing instructions and data to a computer for execution and processing. Examples of suitable non-transitory storage media include a floppy disk, hard disk, optical disk, magneto-optical disk, CD-ROM, CD-R, magnetic tape, non-volatile memory card, ROM, DVD-ROM, Blue-ray disk, solid state disk, and network attached storage (NAS), whether or not such devices are internal or external to the computer. A file containing information can be “stored” on computer readable medium, where “storing” means recording information such that it is accessible and retrievable at a later date by a computer. The computer-implemented method described herein can be executed using programming that can be written in one or more of any number of computer programming languages. Such languages include, for example, Python, Java, Java Script, C, C#, C++, Go, R, Swift, PHP, as well as many others.

The non-transitory computer readable storage medium may be employed on one or more computer systems having a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor, or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as those mentioned above, other high level or low-level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

### Kits

Kits are also provided for carrying out the methods described herein. In some embodiments, the kit includes software for carrying out the computer implemented methods of identifying electrical biomarkers of tumor features, as well as the computer implemented methods of using the identified electrical biomarkers to monitor disease states, develop therapeutic treatments, and/or administer therapeutic treatments for individuals, as described herein. In some embodiments, the kit includes one or more components of a system for carrying out the computer implemented methods to identify exposure to environmental carcinogens, compounds, and other toxins, as described herein.

In addition, the kits may further include, in certain embodiments, instructions for practicing the subject methods. These instructions may be present in the subject kits in a variety of forms, one or more of which may be present in the kit. For example, instructions may be present as printed information on a suitable medium or substrate, e.g., a piece or pieces of paper on which the information is printed, in the packaging of the kit, in a package insert, and the like. Another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Utility

The methods and systems of the present disclosure, e.g., as described above, find use in a variety of applications wherein it is desirable to build a deeper understanding of the systems and methods to identify exposure to environmental carcinogens, compounds, and other toxins in order to develop new therapeutic treatments and improve patient outcomes. In some embodiments, the methods and systems described herein find use wherein it is desirable to identify and characterize the role of environmental carcinogens, compounds, and other toxins, e.g., tumor proliferation and progression in order to enable patient specific tumor characterization, disease monitoring, and therapeutic treatment.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

Aspect 1. A method for training a machine learning model to identify a cellular morphological biomarker (CMB), comprising:
providing a set of histological images to a machine learning model, wherein the set of histological images comprises images of reference tissue and images of test tissue, wherein the test tissue is tissue intentionally exposed to a perturbation, wherein the perturbation comprises at least one chemical compound or at least one source of radiation,
wherein the machine learning model comprises a network layer comprising a set of dictionary elements that represent morphometric features, and the machine learning model identifies a set of CMBs from the images of the reference and test tissues based on the morphometric features.

Aspect 2. The method of Aspect 1, further comprising, prior to providing the set of histological images, generating the images of test tissue, wherein generating the images of test tissue comprises:
exposing tissue to the perturbation to generate the test tissue; and
imaging a sample from the test tissue.

Aspect 3. The method of Aspect 2, further comprising obtaining a biopsy from the test tissue to generate the sample; and optionally staining the sample with hematoxylin and eosin stain.

Aspect 4. The method of any one of Aspects 1-3, wherein the perturbation is selected from a mutagen, a tumor promoter, a tumor anti-promoter, an anti-inflammatory, and ionizing radiation.

Aspect 5. The method of Aspect 4, wherein the ionizing radiation is selected from gamma radiation, alpha particles, beta particles, X-rays, and ultraviolet light.

Aspect 6. The method of Aspect 5, wherein the ionizing radiation is provided by a radiation source.

Aspect 7. The method of Aspect 6, wherein the radiation source is selected from ²³⁵UJ, ²³⁸UJ, ²³⁹Pu, ²³⁸Pu, ²³²Th, ²²⁶Ra, ¹⁴C, ¹³¹I, ¹³⁷Cs, ⁸⁵Kr, ⁵⁶Fe, ⁹⁰Sr, ²¹⁰Po, ⁹⁹Tc, ²²²Rn, and a combination thereof.

Aspect 8. The method of any one of Aspects 1-7, wherein the network layer comprises 4 or fewer, 8 or fewer, 16 or fewer, 32 or fewer, 64 or fewer, 128 or fewer, 256 or fewer, 512 or fewer, 1024 or fewer, or 2048 or fewer dictionary elements.

Aspect 9. The method of any one of Aspects 1-7, wherein the network layer comprises approximately 4, approximately 8, approximately 16, approximately 32, approximately 64, approximately 128, approximately 256, approximately 512, approximately 1024, or approximately 2048 dictionary elements.

Aspect 10. The method of any one of Aspects 1-9, further comprising evaluating the machine learning model to reconstruct a cellular object based on at least one CMB.

Aspect 11. The method of any one of Aspects 1-10, wherein the test tissue is canine, feline, porcine, bovine, equine, human, primate, murine, ovine, or caprine tissue.

Aspect 12. The method of any one of Aspects 1-11, wherein the test tissue is murine tissue. (is this not included in 11?). While redundant, there is a legal strategy for including this in a separate Aspect.

Aspect 13. The method of Aspect 12, wherein the murine tissue is genetically modified to induce a phenotype selected from genome instability, membrane perturbation, increased inflammation, tumor susceptibility, and combinations thereof.

Aspect 14. The method of any one of 1-13, wherein the intentional exposure to the perturbation is in vivo.

Aspect 15. The method of any one of Aspects 1-10, wherein the test tissue is selected from a mouse organoid or a human organoid.

Aspect 16. The method of any one of Aspects 1-13 or 15, wherein the intentional exposure to the perturbation is in vitro.

Aspect 17. The method of any one of Aspects 1-16, wherein the reference tissue and the test tissue are isogenic to one another.

Aspect 18. The method of any one of Aspects 1-17, wherein the set of histological images comprises images of test tissues which were: (a) exposed to different doses of the perturbation, (b) exposed to the perturbation for different lengths of time, and/or (c) exposed to different numbers of doses.

Aspect 19. The method of any one of Aspects 1-18, wherein the machine learning comprises stacked predictive sparse decomposition (SPSD).

Aspect 20. The method of Aspect 19, wherein SPSD comprises:
unsupervised feature learning to identify a collection of dictionary elements; and
spatial pyramid matching the collection of dictionary elements to generate the set of dictionary elements in the network layer of the machine learning model.

Aspect 21. The method of any one of Aspects 1-20, wherein the set of CMBs comprises approximately 5 CMBs, approximately 10 CMBs, approximately 15 CMBs, approximately 20 CMBs, approximately 25 CMBs, approximately 30 CMBs, approximately 50 CMBs, approximately 75 CMBs, approximately 100 CMBs, approximately 150 CMBs, or approximately 200 CMBs.

Aspect 22. The method of any one of Aspects 1-21, wherein the method further comprises identifying a subset of CMBs, from the set of CMBs, that significantly correlate with the images of the test tissue versus the images of the reference tissue.

Aspect 23. The method of Aspect 22, wherein identifying said subset of significantly correlated CMBs comprises: identifying CMBs with the largest variances in abundance contributing to 95% or more of total data variation, and identifying those that are predictive of test tissue versus reference tissue.

Aspect 24. The method of Aspect 23, wherein the identification of those CMBs that are predictive comprises step-wised multivariate linear regression.

Aspect 25. The method of any one of Aspects 22-24, wherein the method further comprises generating a CMB perturbation scoring algorithm based on the identified subset of significantly associated CMBs.

Aspect 26. The method of Aspect 25, wherein the CMB perturbation scoring algorithm has the formula:

$$CMB perturbation score = Intercept + {\sum }_{i = 1}^{N} ( coefficient of {CMB}^{i} ) * ( {CMB}^{i} )$$

wherein “N” is the number of CMBs in the identified subset of significantly associated CMBs, and the coefficient of CMBⁱ is obtained for each significantly associated CMB using step-wised multivariate linear regression.

Aspect 27. A method of identifying physiological effects of a compound, comprising:
providing one or more histological images of one or more tissue samples from an individual to a machine learning model trained by the method of any one of Aspects 22-26,
wherein the trained machine learning model, based on the one or more histological images from the individual, outputs CMB values for the subset of significantly correlated CMBs.

Aspect 28. The method of Aspect 27, further comprising calculating a CMB perturbation score for the individual using the CMB perturbation scoring algorithm.

Aspect 29. The method of Aspect 28, determining a diagnosis and/or prognosis based on the CMB perturbation score, optionally wherein the diagnosis is a cancer-related diagnosis and/or the prognosis is a cancer-related prognosis.

Aspect 30. The method of Aspect 28 or 29, wherein the CMB perturbation score indicates an exposure risk for the individual for exposure to a harmful compound.

Aspect 31. The method of Aspect 30, wherein the exposure risk indicates a risk of developing cancer.

Aspect 32. The method of Aspect 28 or 29, wherein the CMB perturbation score indicates a record of past exposure to a compound.

Aspect 33. The method of any one of Aspects 27-32, wherein the one or more tissue samples are obtained from a biopsy.

Aspect 34. The method of any one of Aspects 27-33, wherein the individual is a canine, a feline, a porcine, a bovine, an equine, a human, a primate, a murine, an ovine, or a caprine.

Aspect 35. The method of any one of Aspects 27-34, wherein the individual is human, and the machine learning model is trained from a set of histological images from a mouse.

Aspect 36. The method of any one of Aspects 27-35, wherein the individual is suspected of having been exposed to a perturbation.

Aspect 37. The method of Aspect 36, wherein it is unknown to which perturbation the individual is suspected of having been exposed.

Aspect 38. The method of Aspect 37, wherein the method further comprises identifying, based on the CMB perturbation score, the perturbation to which the individual was exposed.

Aspect 39. The method of any one of Aspects 28-38, further comprising determining a mediation recommendation.

Aspect 40. The method of Aspect 39, wherein the mediation recommendation is selected from environmental remediation and medical intervention.

Aspect 41. The method of Aspect 40, wherein the medical intervention comprises excision, chemotherapy, immunotherapy, radiotherapy, or any combination thereof.

Aspect 42. The method of Aspect 40, wherein the environmental remediation comprises replacing pipes, phytoremediation, topsoil replacement, increasing water purity, increasing air circulation, increasing air purity, sequestering or encapsulating lead paint, or any combination thereof.

Aspect 43. A method for generating a reference library for cellular morphological biomarkers (CMBs), comprising:
exposing a collection of murine tissue to a perturbation, wherein the perturbation comprises at least one chemical compound or at least one source of radiation, and each piece of tissue in the collection is acutely exposed or chronically exposed to the perturbation; and
obtaining a histological image of each piece of tissue in the collection and at least one reference piece of murine tissue which has not been exposed to a perturbation.

Aspect 44. The method of Aspect 43, wherein the murine tissue comprises in vivo tissue on mice.

Aspect 45. The method of Aspect 43 or 44, wherein the murine tissue comprises isogenic mice, inbred mice, heterogeneous mice, and/or genetically modified mice.

Aspect 46. The method of any one of Aspects 43-45, wherein the murine tissue comprises tissue from primary tumors.

Aspect 47. The method of Aspect 46, wherein the primary tumors comprise: early stage benign lesions, malignant carcinomas, and/or metastases.

Aspect 48. The method of any one of Aspects 43-47, wherein the perturbation is selected from a mutagen, a tumor promoter, a tumor anti-promoter, an anti-inflammatory, a chemotherapeutic, an immunotherapeutic, and ionizing radiation.

Aspect 49. The method of any one of Aspects 43-48, further comprising sequencing the exposed tissue and the at least one reference tissue, wherein sequencing comprises single cell RNA sequencing, bulk RNA sequencing, and/or DNA sequencing.

Aspect 50. The method of any one of Aspects 43-49, wherein tissue in the collection: (a) exposed to different doses of the perturbation, (b) exposed to the perturbation for different lengths of time, and/or (c) exposed to different numbers of doses.

Aspect 51. The method of any one of Aspects 43-50, further comprising providing the obtained histological images to a machine learning model, such that the machine learning model identifies a morphometric profile comprised of a set of CMBs to differentiate each tissue type and treatment.

Aspect 52. A method for screening compounds for medical efficacy, comprising:
providing one or more histological images of one or more tissue samples, wherein each tissue sample has been exposed to a compound to a machine learning model trained by the method of any one of Aspects 22-26 or 51,
wherein the trained machine learning model, based on the one or more histological images, outputs a physiologic response prediction for the compound.

### EXPERIMENTAL EXAMPLES

The following examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like

### Example 1: Machine Learning Identifies Effects of Low Dose Radiation Exposure in Mice that Predict Genome Instability and Prognosis of Human Cancers

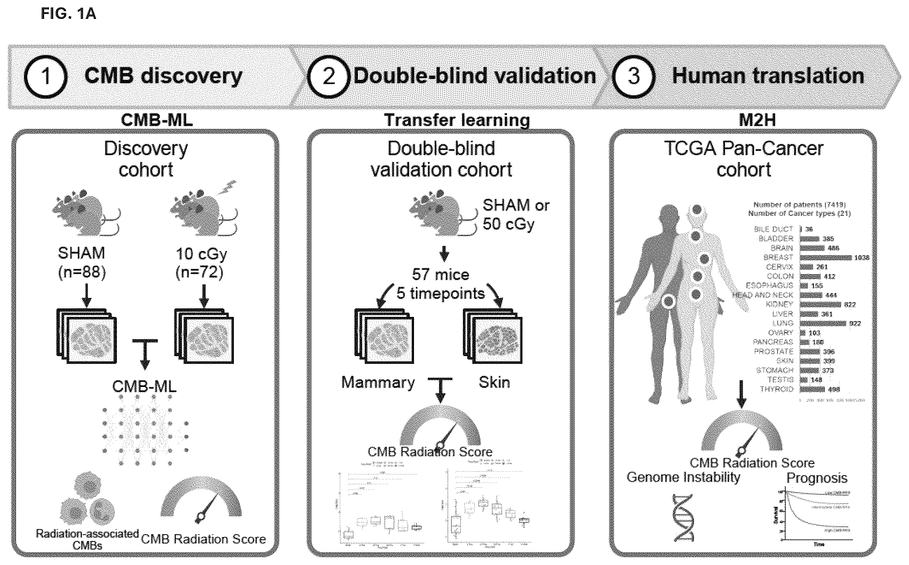

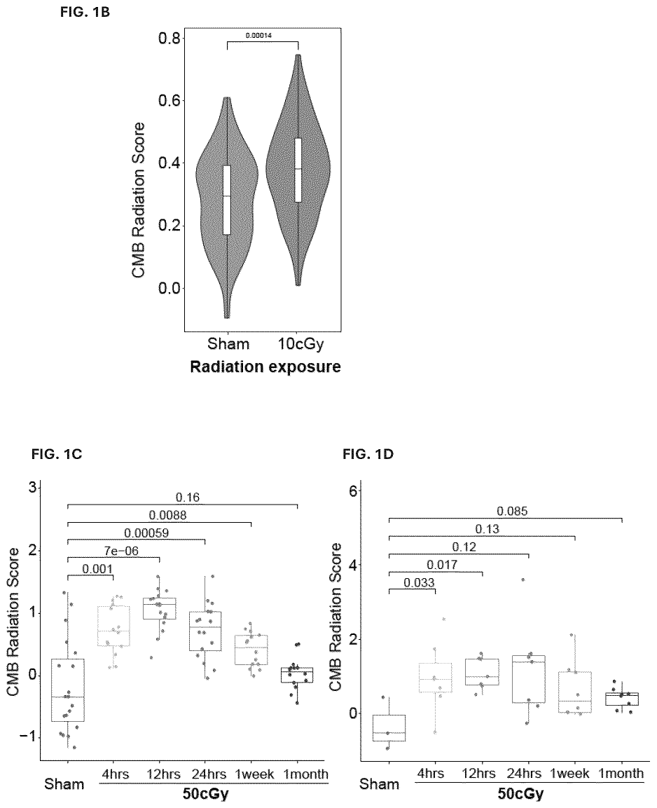

The associations between radiation exposure and cancer are well known, but whether the cancers developing because of radiation exposure are indistinguishable from those that occur naturally remains largely unclear. Therefore, searching for biosignatures of ionizing radiation exposure is still an active area of radiation research. In this study, an established artificial intelligence (CMB-ML) pipeline¹⁻³ was used to discover cellular morphometric biomarkers (CMBs) for radiation exposure from histological whole-slide-images (WSIs) with haematoxylin and eosin (H&E) staining (FIG. 1a).

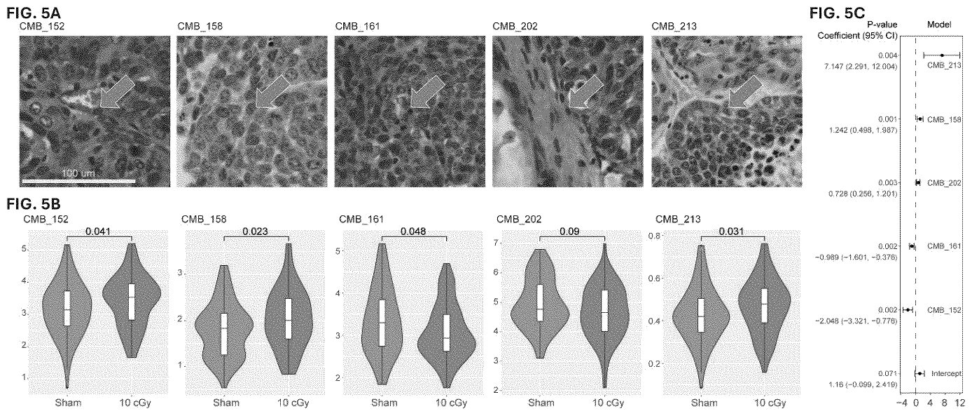

The training cohort contains 160 tumor samples (88 from sham-treated mice and 72 from 10 cGy whole body x-ray irradiated mice) (FIG. 1a), which were obtained from a previous study⁴. The CMB-ML pipeline was applied to identify cellular objects, each of which was represented by 15 morphometric properties as described in previous work¹, and to profile the underlying CMBs in WSIs with H&E staining. It was found that 5 CMBs were significantly associated with radiation exposure (FIG. 5). Multivariate analysis revealed that 5 CMBs had independently predictive value to radiation exposure. Then, a CMB radiation scoring system was established based on the abundance of these 5 CMBs (details see methods). Expectedly, the CMB radiation scores are significantly different between irradiated and sham-treated samples (p=0.00014, FIG. 1b).

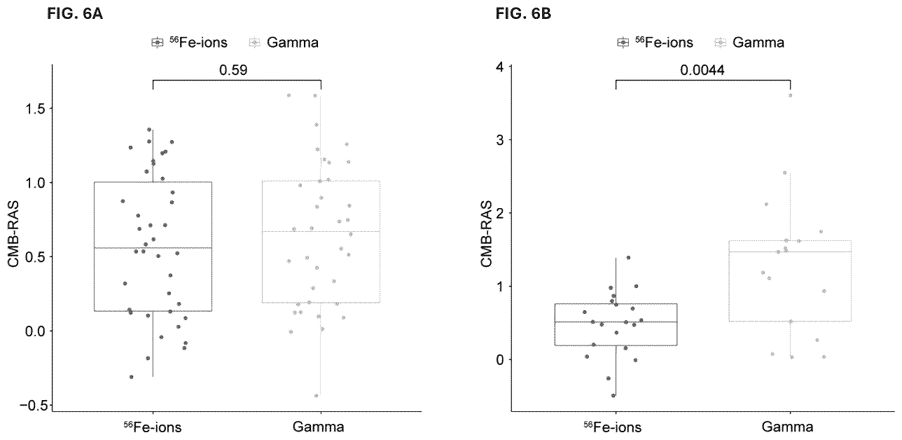

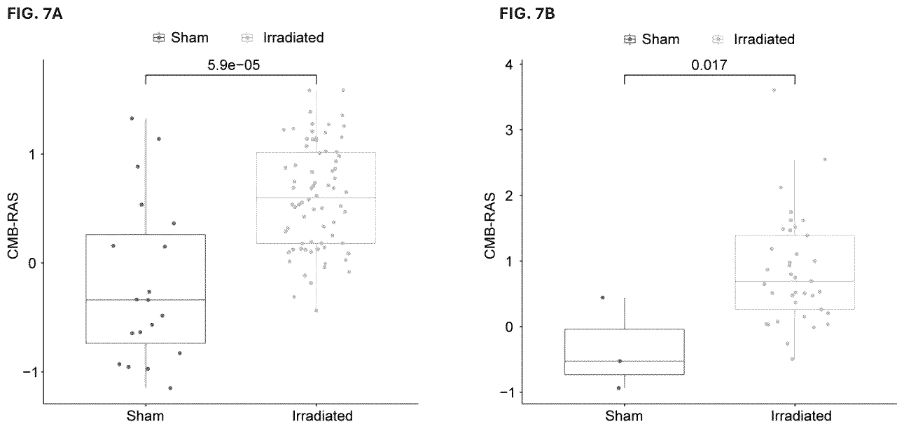

To validate CMB radiation score, the pre-established CMB radiation scoring system was applied in the training cohort to a double-blind mouse cohort where normal skin and mammary gland were collected from mice at different timepoints post 50 cGy gamma or ⁵⁶Fe-ions irradiation exposure (FIG. 1a). It was first found that CMB radiation scores are not significantly different between gamma and ⁵⁶Fe-ions irradiation exposure in skin at different time points (FIG. 6a), where CMB radiation scores tended to be higher in mammary gland with gamma irradiation exposure comparing to these with ⁵⁶Fe-ions irradiation exposure (FIG. 6b). In the following study, the samples from gamma and ⁵⁶Fe-ions irradiation exposure were pooled for remaining analyses. The CMB radiation scores showed significant differences between radiation-exposed and sham-treated groups in both skin and mammary tissues (FIG. 7), indicating that the radiation-associated cellular morphometric changes discovered from tumors even exists in normal tissues. Moreover, we found that the CMB radiation scores changed in a time-dependent manner in both skin and mammary gland (FIGS. 1b and 1c).

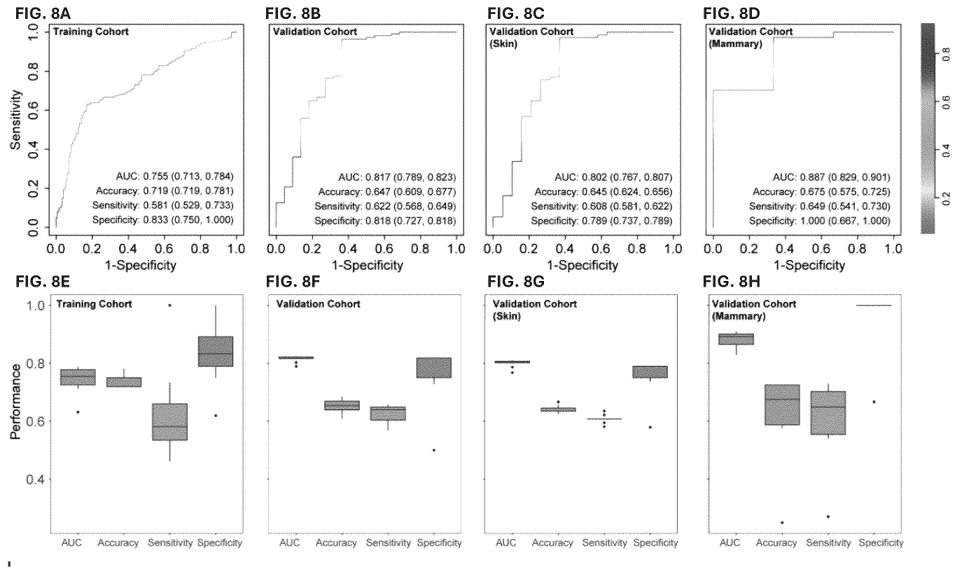

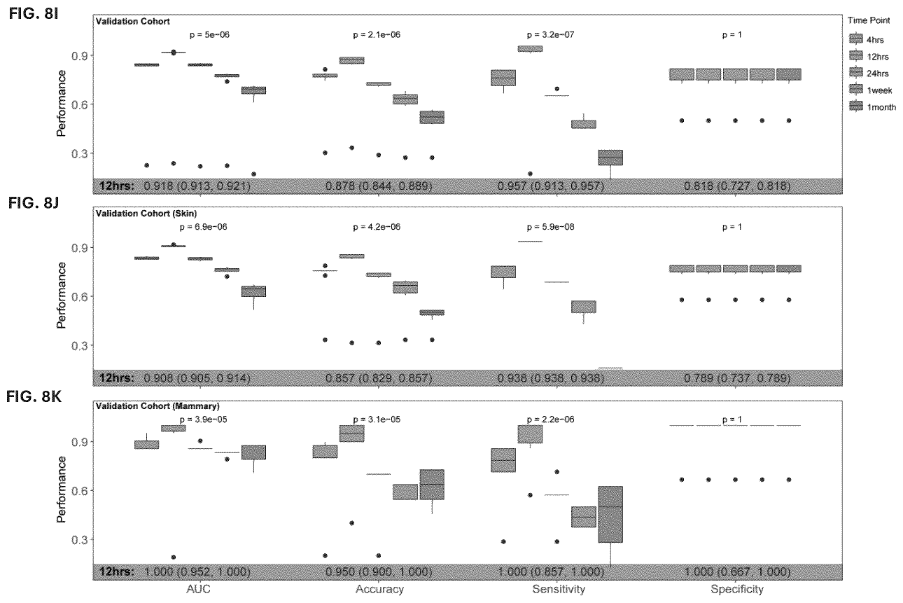

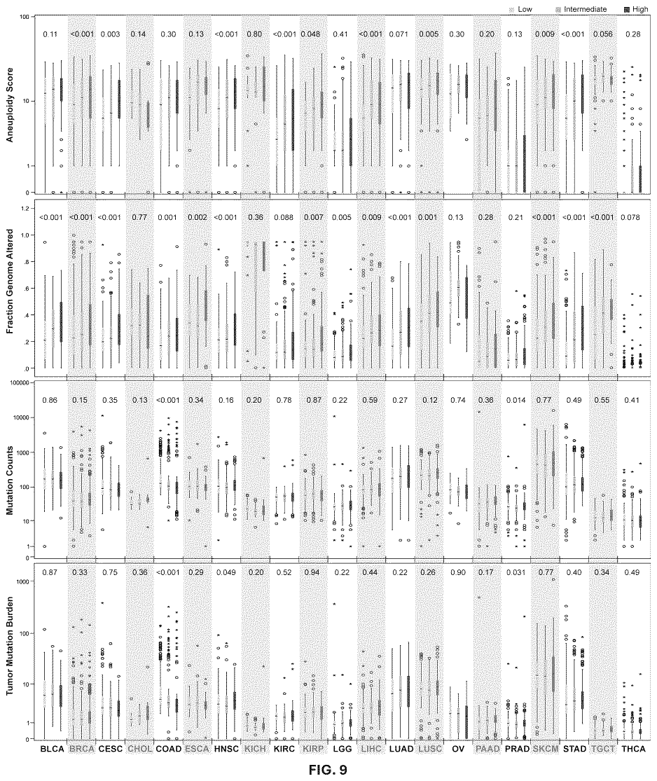

To validate the predictive power of CMBs in distinguishing samples with/without radiation exposure, a classification system was established using a LASSO and bootstrapping strategy. The evaluation on hold-out samples during bootstrapping showed that the 5 CMBs have the power of predicting radiation exposure (FIG. 8a, e; AUC: 0.755 [0.713, 0.784]; Accuracy: 0.719 [0.719, 0.781]; Sensitivity: 0.581 [0.529, 0.733]; Specificity: 0.833 [0.750, 1.000]). The validation of the classification system confirmed the predictive power of these CMBs on the entire validation cohort (FIG. 8b, f; AUC: 0.817 [0.789, 0.823]; Accuracy: 0.647 [0.609, 0.677]; Sensitivity: 0.622 [0.568, 0.649]; Specificity: 0.818 [0.727, 0.818]), normal skin tissues (FIG. 8c, g; AUC: 0.802 [0.767, 0.807]; Accuracy: 0.645 [0.624, 0.656]; Sensitivity: 0.608 [0.581, 0.622]; Specificity: 0.789 [0.737, 0.789]) and normal mammary glands (FIG. 8d, h; AUC: 0.887 [0.829, 0.901]; Accuracy: 0.675 [0.575, 0.725]; Sensitivity: 0.649 [0.541, 0.730]; Specificity: 1.000 [0.667, 1.000]). Expectedly, the predictive power in normal mammary glands exceeds the predictive power in normal skin tissues, given the CMBs were originally learned from mouse mammary tumors. Moreover, the predictive power changed was found to change in a time-dependent manner in both skin and mammary gland, which is consistent with the trend of the CMB radiation scores (FIGS. 1b and 1c; FIG. 8i-k;). Specifically, when the CMB radiation scores peaked around 12 hrs after radiation exposure, the maximum predictive power was also observed in the entire validation cohort (AUC: 0.918 [0.913, 0.921]; Accuracy: 0.878 [0.844, 0.889]; Sensitivity: 0.957 [0.913, 0.957]; Specificity: 0.818 [0.727, 0.818]), normal skin tissues (AUC: 0.908 [0.905, 0.914]; Accuracy: 0.857 [0.829, 0.857]; Sensitivity: 0.938 [0.938, 0.938]; Specificity: 0.789 [0.737, 0.789]) and normal mammary glands (AUC: 1.000 [0.952, 1.000]; Accuracy: 0.950 [0.900, 1.000]; Sensitivity: 1.000 [0.857, 1.000]; Specificity: 1.000 [0.667, 1.000]).

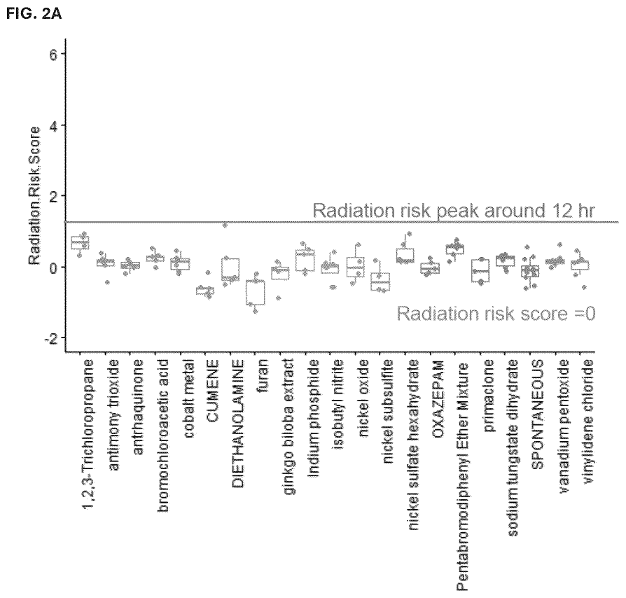

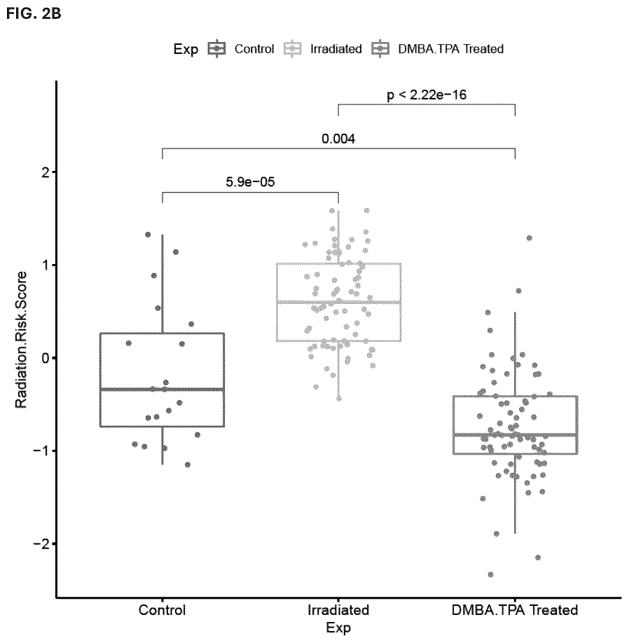

To examine whether the CMB radiation scores specified to radiation, the pre-established CMB radiation scoring system was applied to tumors induced by 19 different chemicals. The CMB radiation scores in these tumors were found to not differ from these in spontaneous tumors (FIG. 2a). Furthermore, no significant difference in the CMB radiation scores was found between normal control skin and DMBA-TPA treated skin (FIG. 2b).

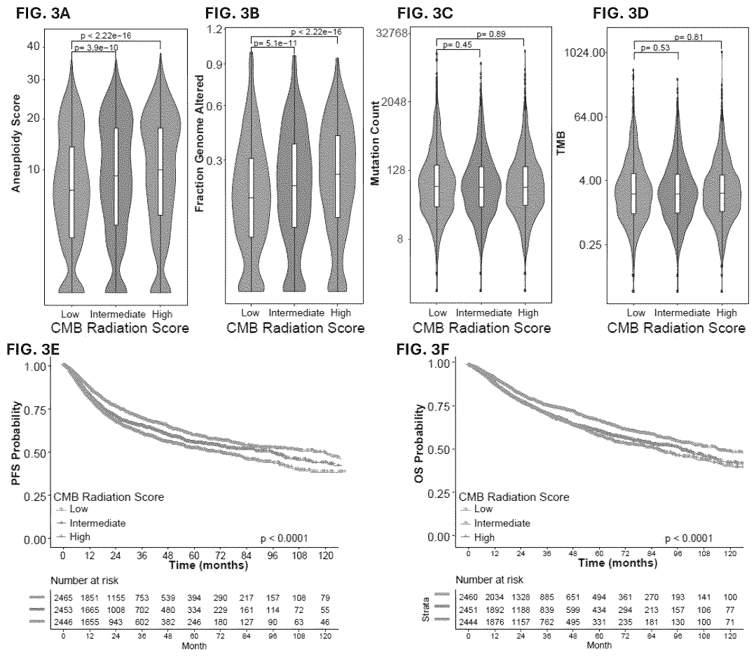

To decode human cancer with five radiation-associated CMBs and the CMB radiation score, which allow the inference of the contribution of radiation to human cancer, these CMBs were identified and quantified from 7,419 patients across 21 cancer types in TCGA pan-cancer cohort (FIG. 1a, Table 1) using a previously established transfer learning pipeline 5, then used the pre-built the CMB radiation score formula in the mouse cohort to calculate the CMB radiation scores for each patient, and divided the patients into three groups (High=top tertile, intermediate=middle tertile, and low=bottom tertile) based on the CMB radiation scores. At the pan-cancer level, the CMB radiation scores are significantly and positively correlated to aneuploidy score and fraction of genome altered, while does not correlate to mutational burden in human cancer (FIG. 3a-3d). Moreover, the patients with high CMB radiation scores had a significantly shorter progression-free and overall survival (FIGS. 3e and 3f). At cancer-type-specific level, the CMB radiation scores are significantly and positively correlated to aneuploidy score and fraction of genome altered in a majority of cancer types (FIG. 9). The CMB radiation scores are not significantly correlated to mutation counts and burden in majority of cancer types, but unexpectedly, the CMB radiation scores are significantly and negatively correlated to mutation counts and burden in colon and prostate adenocarcinomas (FIG. 9).

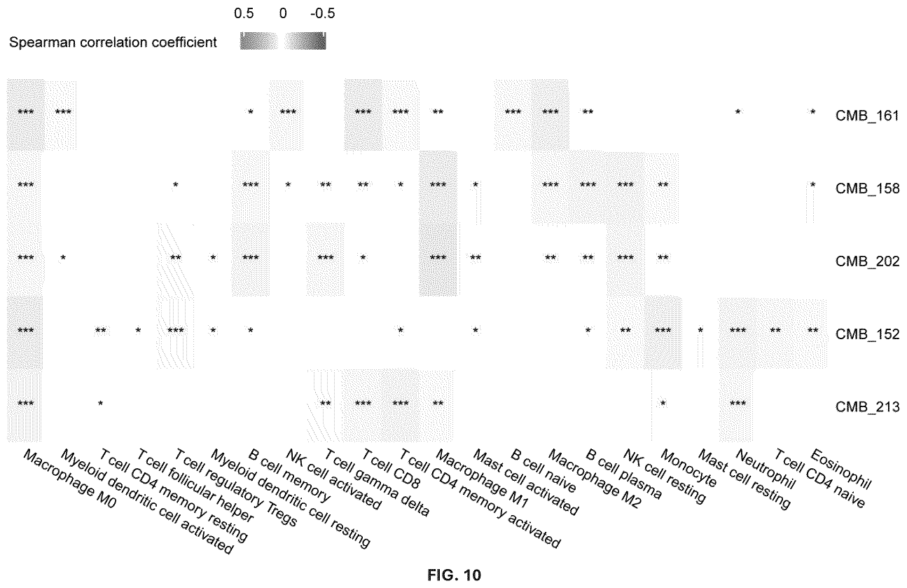

High CMB radiation scores are significantly associated with poor prognosis regarding overall or progression-free survival in 14 of 21 cancer types including cancer from bladder, breast, cervix, esophagus, kidney, liver, ovary, pancreas, skin, stomach and testis (Table 2). The associations between radiation exposure and cancer have been reported in these organs. In addition, the tumor microenvironment (TME), including diverse immune cell types, cancer-associated fibroblasts, endothelial cells, etc., plays an important role in prognosis and treatment response. The significant association between radiation associated CMBs and tumor immune microenvironments at pan-cancer level (FIG. 10) explains the prognostic value of the CMB radiation scores.

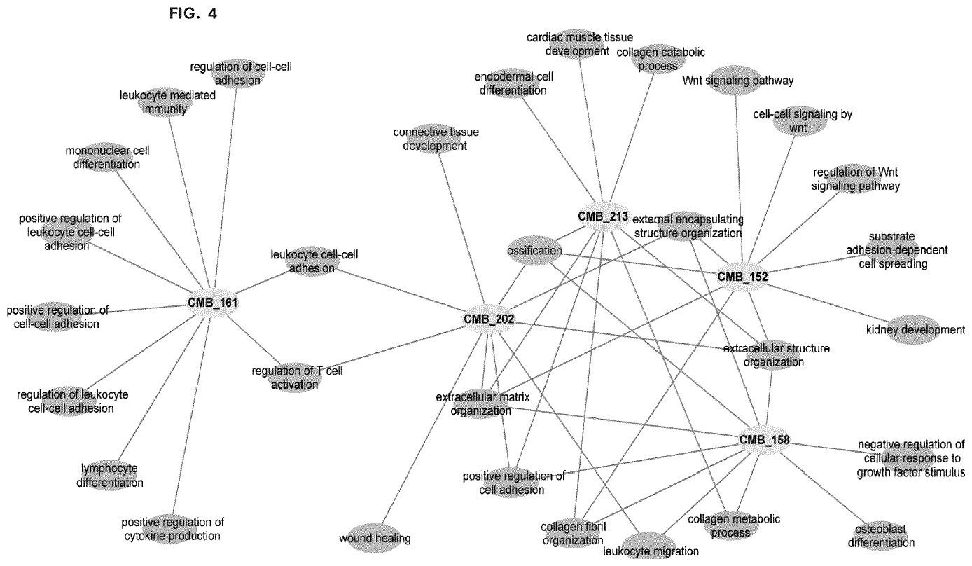

To explore the underlying molecular association of CMB in human pan-cancer, a CMB-Enrichment-Network was constructed based on CMB-associated genes in human pan-cancer with respect to Biological Process (BP), Cellular Component (CC), Molecular Function (MF) and Kyoto Encyclopedia of Genes and Genomes (KEGG). Specifically, CMB-BP-Network (FIG. 4), CMB-CC-Network (FIG. 10a), CMB-MF-Network (FIG. 10b) and CMB-KEGG-Network (FIG. 10c) were constructed.

### Methods

### Mice in Training Cohort

Animal treatment and care was carried out in accordance with the animal protocols and approved by the Animal Welfare and Research Committee at Lawrence Berkeley National Laboratory. SPRET/EiJ mice were obtained from Jackson Laboratories (Bar Harbor, Maine). The female interspecific F1 hybrid mice between BALB/c, a strain susceptible to radiation-induced mammary tumor development and SPRET/EiJ, a resistant strain, were crossed with male BALB/c to generate F1 backcross (F1Bx) mice (BALB/c×SPRET/EiJ)×BALB/c. The inguinal fat pad of F1Bx host mice were divested of endogenous mammary epithelium at 3 weeks of age at 10-11 weeks of age, half of the mice received 10 cGy of whole-body radiation 3 days prior to transplantation of the inguinal mammary glands with Trp53 null fragments. Mice were monitored and palpated for mammary tumor development for 18 months. At the time of dissection, the mammary glands were excised, and formalin fixed. H&E staining and digital scanning (at 40×) was generated by the UCSF Helen Diller Family Comprehensive Cancer Center Mouse Pathology Core.

### Mice in Double-Blind Validation Cohort

### Patients

7,419 cancer patients across 21 cancer types with both whole slide diagnostic slides and clinical information available were included in the TCGA pan-cancer cohort. Specifically, this pan-cancer cohort consisted of human cancer patients from TCGA-BLCA (n=385), TCGA-BRCA (n=1038), TCGA-CESC (n=261), TCGA-CHOL (n=36), TCGA-COAD (n=412), TCGA-ESCA (n=155), TCGA-HNSC (n=444), TCGA-KICH (n=65), TCGA-KIRC (n=490), TCGA-KIRP (n=267), TCGA-LGG (486), TCGA-LIHC (n=361), TCGA-LUAD (n=461), TCGA-LUSC (n=461), TCGA-OV (n=103), TCGA-PAAD (n=180), TCGA-PRAD (n=396), TCGA-SKCM (n=399), TCGA-STAD (n=373), TCGA-TGCT (n=148), TCGA-THCA (n=498). Diagnostic slides were downloaded from Genomic Data Commons Data Portal (portal.gdc.cancer.gov/), and clinical and molecular data were downloaded from cBioPortal (www.cbioportal.org/).

Identification of Cellular Morphometric Biomarkers (CMBs) and Development of the CMB Radiation Associated Score from the Training Cohort

The information from the H&E-stained digital slides were processed and used to develop the CMBs and the CMB radiation scores. Based on the stacked predictive sparse decomposition (SPSD) technique and our cellular morphometric subtyping via machine learning (CMB-ML) pipeline [19-21], 256 CMBs were defined from cellular objects extracted from the whole slide images (WSI) of H&E stained tissue histology sections in our training cohort. In the CMB-ML pipeline, a single network layer with 256 dictionary elements (i.e., CMBs) was used and a sparsity constraint of 30 at a fixed random sampling rate of 1000 cellular objects per WSIs from the cohort. The pre-trained SPSD model reconstructed each cellular region as a sparse combination of pre-defined 256 CMBs and thereafter represents each sample as an aggregation of all delineated cellular objects belonging to the same mouse. The experimental settings was identical to our previous study to keep the reconstruction error less than 10% during training.

Digital slides corresponding to sham and irradiated status were set to a radiation score of 0 and 1.0, respectively. And the top 64 CMBs, with the largest variances in abundance contributing to 99.6% of total data variation, were then deployed in a step-wised multivariate linear regression process (R, version 3.6.0), where 5 out of these 64 CMBs were selected after step-wised model optimization. The construction of the CMB radiation score was defined below, where the coefficients of the final N=5 CMBs were obtained from step-wised multivariate linear regression analysis:

$$CMB perturbation score = Intercept + {\sum }_{i = 1}^{N} ( coefficient of {CMB}^{i} ) * ( {CMB}^{i} )$$

### Double-Blind Validation of the CMB Radiation Scores in an Independent Mouse Cohort

Pre-trained CMBs were extracted and pre-established CMB radiation score system was applied to the double-blinded digital slides in a validation cohort from UCSF, where mouse skin and mammary tissues were collected at five time points (i.e., 4 hrs, 12 hrs, 24 hrs, 1 week and 1 month) post 50 cGy gamma or ⁵⁶Fe-ions radiation. The CMB radiation scores were then compared across experimental factors (e.g., sham vs irradiation and gamma vs ⁵⁶Fe-ions) and time points via non-parametric Mann-Whitney test in R (version 3.6.0).

### Construction and Validation of Machine Learning Model for Radiation Exposure Prediction

Least Absolute Shrinkage and Selection Operator (LASSO) machine learning algorithm (glmnet package in R, version 4.1.4) was deployed with pre-determined 5 CMBs through bootstrapping strategies at fixed sampling ratio (i.e., 80% training and 20% hold-out testing) and 100 iterations on the training cohort. The top 10 LASSO models with the best hold-out testing performance formed as an assembled-classifier (i.e., all 10 LASSO models were used for the radiation status prediction, and the final decision was aggregated through all LASSO models). The assembled classifier was then validated on the double-blind validation cohort across tissue types and time points.

### Translational Study of the CMB Radiation Scores in Human Pan-Cancer

Pre-trained CMBs were extracted and pre-established CMB radiation score system were applied to the diagnostic slides of 7,419 cancer patients across 21 cancer types. Patients per cancer type were divided into three groups (High=top tertile, intermediate=middle tertile, and low=bottom tertile) based on the CMB radiation scores. Genome instability in term of aneuploidy score and fraction of genome altered, mutation counts, mutation burden and prognosis (i.e., overall survival and progression-free survival) were then evaluated at pan-cancer level among different the CMB radiation score groups.

### Exploration of Underlying Tumor Microenvironments (TMEs) Association of CMB in Human Pan-Cancer

The CIBERSORT estimation of the TMEs (i.e., abundances of member cell types in a mixed cell population) was downloaded from public website (timer.cistrome.org/), and the association between CMBs and TMEs was calculated via spearman correlation, and represented by a heatmap (ComplexHeatmap package in R, version 3.18).

### Exploration of Underlying Molecular Association of CMB in Human Pan-Cancer

CMB-Enrichment-Network study was performed based on the following steps: (1) significantly CMB-associated genes were selected per cancer type per CMB (spearman correlation, |correlation coefficient|>0.15 and p<0.05, R version 3.6.0); (2) pre-selected CMB-associated genes with selection-frequency >20% (i.e., genes were co-selected at least in 5 out of 21 cancer types) were identified as CMB-associated pan-cancer genes per CMB; (3) Enrichment analysis (i.e., BP/Biological Process, CC/Cellular Component, MF/Molecular Function, and KEGG/Kyoto Encyclopedia of Genes and Genomes) was performed (clusterProfiler package in R, version 4.1.0) on CMB-associated pan-cancer genes per CMB; and (4) CMB-BP-Network, CMB-CC-Network, CMB-MF-Network and CMB-KEGG network were then constructed and visualized in Cytoscape (version 3.8.2).

### Statistical Analysis

The Chi-square test was used to test significance when category variables were compared between groups. Mann-Whitney tests and Kruskal-Wallis tests were used when continuous variables were compared among two groups or more than two groups, respectively. Prognosis was estimated by Cox proportional hazards regression. All statistical analysis was performed in R (version 3.6.0), and p<0.05 was considered as statistically significant.

### REFERENCES

1 Liu, X.-P. et al. Clinical significance and molecular annotation of cellular morphometric subtypes in lower-grade gliomas discovered by machine learning. Neuro-Oncology, noac154, doi: 10.1093/neuonc/noac154 (2022).
2 Chang, H. et al. From Mouse to Human: Cellular Morphometric Subtype Learned From Mouse Mammary Tumors Provides Prognostic Value in Human Breast Cancer. Frontiers in Oncology 11, doi: 10.3389/fonc.2021.819565 (2022).
3 Mao, X. Y. et al. iCEMIGE: Integration of CEII-morphometrics, Microbiome, and GEne biomarker signatures for risk stratification in breast cancers. World J Clin Oncol 13, 616-629, doi: 10.5306/wjco.v13.i7.616 (2022).
4 Zhang, P. et al. Identification of genetic loci that control mammary tumor susceptibility through the host microenvironment. Sci Rep 5, 8919, doi: 10.1038/srep08919 (2015).
5 Chang, H. et al. From Mouse to Human: Cellular Morphometric Subtype Learned From Mouse Mammary Tumors Provides Prognostic Value in Human Breast Cancer. Front Oncol 11, 819565, doi: 10.3389/fonc.2021.819565 (2021).

### Example 2: CMB Analysis of Tumor Promoters and Mutagens

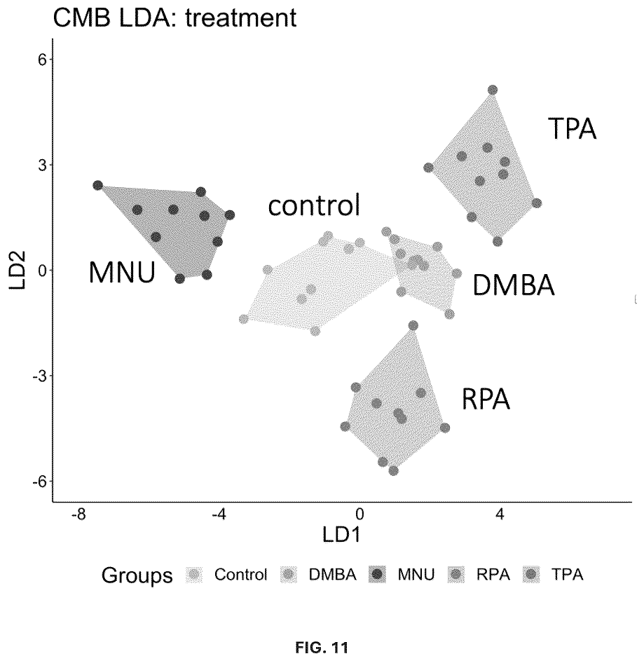

In this example, mice were treated on the skin with a series of chemicals that are known to act as strong or weak tumor promoters, or as mutagens. CMB analysis of skins treated with these different agents shows that they can be discriminated clearly from each other, as shown by LDA analysis of the CMB patterns. Specifically, mice were treated with the mutagens methylnitrosourea (MNU) or dimethylbenzanthracene (DMBA), or with a strong promoter (Tetradecanoyl-phorbol-acetate, TPA) or a weak promoter (Retinoyl-phorbol acetate RPA). As illustrated in FIG. 11, AI-analyzed scans of histological sections showed minimal overlap between these different treatments based on 10 samples for each category.

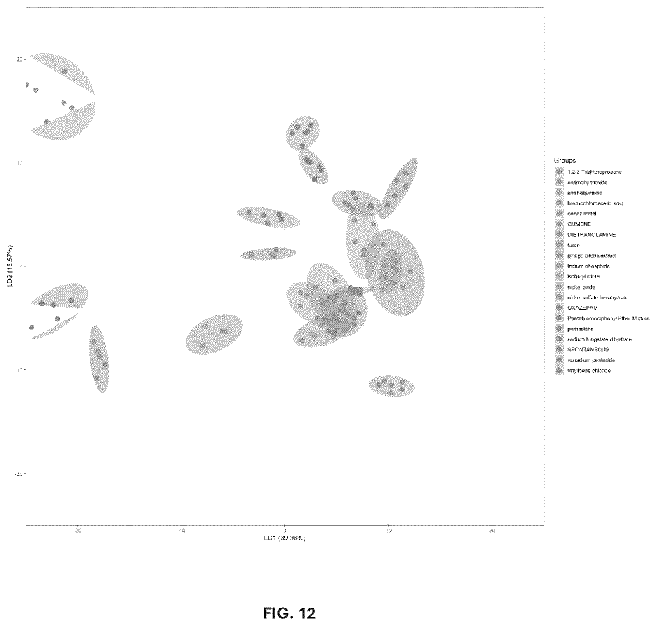

Next, histological sections from mice that developed liver tumors after treatment with a series of known or suspected human carcinogens (FIG. 12). Specifically, the mice were treated (at National Toxicology Program, NIEHS) chronically for 2 years with the series of chemicals shown in FIG. 12, all of which are known or suspected of causing cancer in humans. Only 2 of these chemicals showed clear signs of being mutagenic. A control group developed spontaneous tumors which have a longer latency. The CMBs for most chemicals fell into distinct groups, although with some overlap, in spite of the fact that some groups had only 4 animals. LDA analysis also showed different patterns for most of these carcinogens, indicating that many have distinct effects on tumor morphology, although some may overlap.

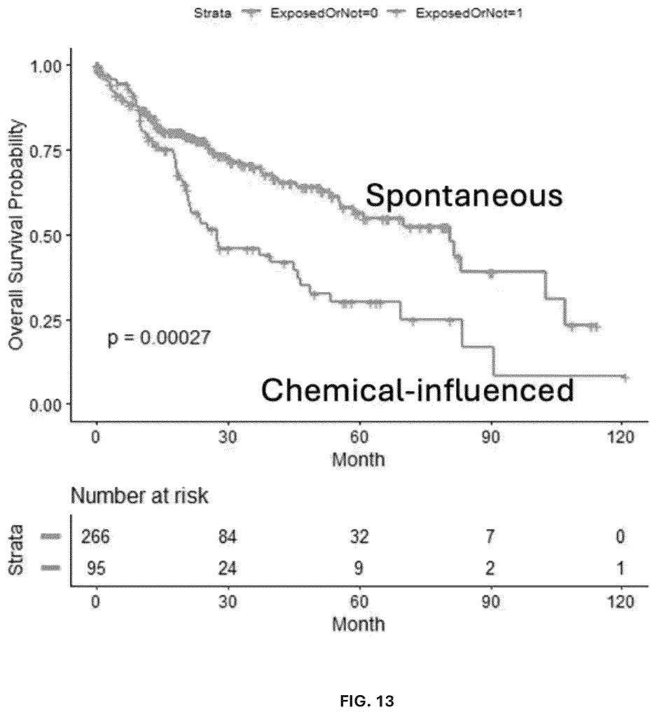

In order to test the validity of CMBs in human samples, liver tumors from TCGA were investigated, for which digitized scans of histological sections were available. This analysis clearly detected the same CMBs in human liver tumors, and an aggregated score for the presence of CMBs for any of the cancer-causing chemicals was predictive of patient survival, independent of other variables normally associated with outcome (FIG. 13).

The data shown in FIGS. 11-13 indicate that AI scanning of mouse tumor sections using the CMB algorithm can discriminate between exposure to different types of causative factors including mutagens and tumor promoters. Moreover, CMBs indicative of chemical exposures in mouse samples are also conserved in human tumors, where they are strongly associated with patient survival.

Finally, an analogous study was carried out to investigate the CMBs linked to a different type of known human carcinogen, radiation, in both radiation-induced tumors and in normal tissue (see Example 1). Tumors were induced in mice by implantation of p53 null mammary cells, which give rise to tumors after exposure to a low dose of gamma radiation (10cGy). CMB analysis of tumor histological sections identified a set of radiation-associated CMBs that were only present in radiation-exposed tumors. A CMB Radiation Score was established and tested this set of CMBs in a blinded study of normal mouse mammary gland and skin after a single exposure to 50cGy gamma radiation. This showed that the CMB radiation score was induced in a time-dependent fashion after exposure to this low radiation dose. The mouse radiation CMBs in tumors were quantified from 7,419 patients across 21 cancer types in a TCGA pan-cancer cohort and found that the CMB radiation score was significantly and positively correlated to genomic instability, in particular to aneuploidy and fraction of genome altered, while it did not correlate with point mutational burden. Patients with a high CMB radiation score also had a significantly shorter progression-free and overall survival.

### Example 3: Linking CMBs to Gene Expression

Gene expression data was obtained from tissues used in histological images for training. This analysis was possible in a set of samples for which the tissue (either normal or tumor) was available for both transcriptomic analysis and histological imaging that can be scanned to identify specific CMBs. The variation in the score for specific CMBs across the sample set enabled a direct correlation with expression of all genes in the genome. Some CMBs identify cell cycle components, and some others identify extracellular matrix components. Preliminary data suggest that some CMBs recognize what were previously called Metagenes. With this type of data CMB scores will have directly actionable consequences, linking to pathways and individual druggable genes for specific CMBs.

### Example 4: Cellular Morphometric Biomarkers can Distinguish Between Different Chemical Exposures

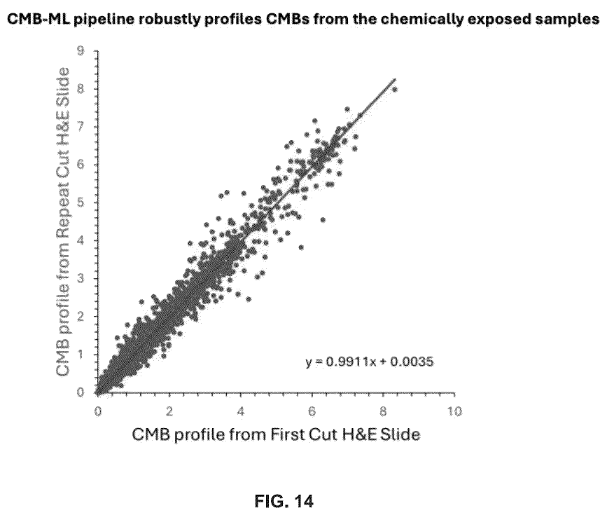

FIG. 14. A previous study on Cellular Morphometric Biomarkers from mouse skin samples treated with a range of chemicals, including DMBA, TPA, MNU, and RPA, showed that LDA analysis could discriminate between samples treated with each chemical. The data here show a technical replicate of this experiment, using additional histological sections cut from the same sample set. The profiles obtained from this second analysis are almost perfectly correlated with the first data set, showing extremely high replication of the ability to discriminate between chemical exposures using independent sections from the same samples.

### Example 5: Replication of CMB Profiles Using Independent Samples

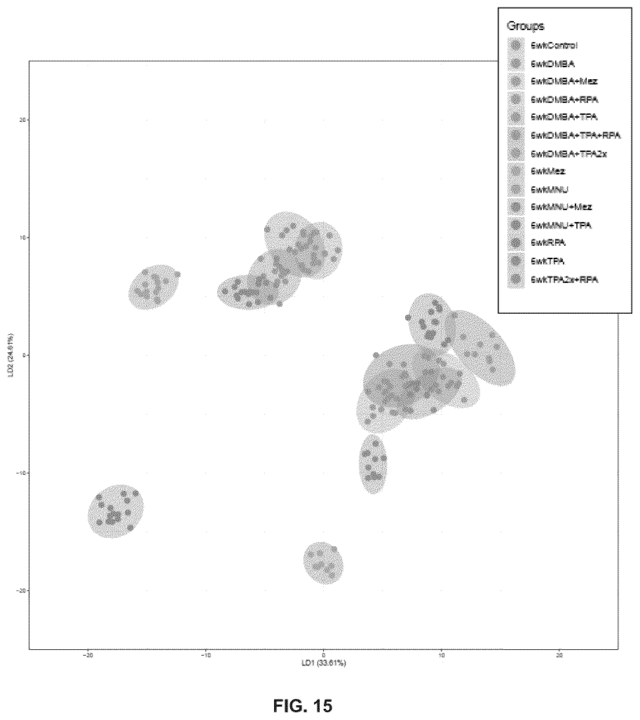

FIG. 15. A new study was carried out to identify CMBs in an additional set of samples from mouse skin treated with a range of chemicals, in this case including combinations of some of the previous chemical classes. Although all of these treatments caused inflammation, LDA analysis clearly distinguished many of the different treatment groups.

### Example 6: Validation of Selected CMBs Derived from an Independent Set of Samples of Skin Squamous Carcinomas and Matched Normal Skin Samples

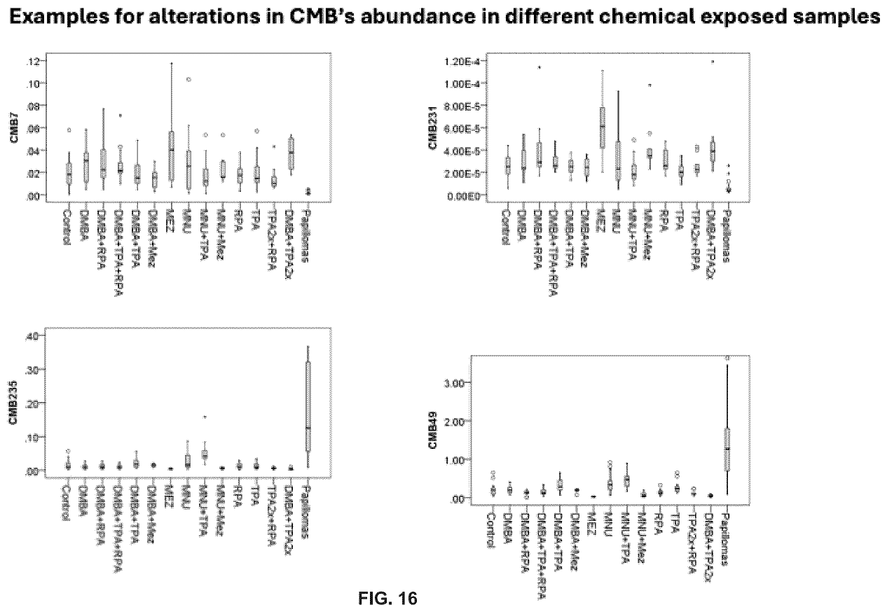

FIG. 16. A set of 256 CMBs was generated de novo using scans of several hundred normal mouse skin and carcinoma samples induced by DMBA/TPA exposure. Analysis of this cohort identified opposite relationships between several CMBs that were either higher in carcinomas than in normal tissue, or vice versa. For example, CMB49 and CMB235 were strongly positive in carcinomas but low in normal tissue, which CMB7 and CMB231 showed the oppositive patterns. Scanning of normal mouse skin and a small cohort of papillomas showed that CMBs 235 and 49 were highest in these transformed papilloma samples, but lowest in samples treated with Mezerein. Exactly the opposite was shown for CMBs 7 and 231, which had the lowest scores in papillomas and the highest after mezerein treatment. These data were supported by gene expression analysis, which showed that CMBs 49 and 235 were positively correlated with expression of genes linked to cell growth, DNA replication and protein translation, while CMBs 7 and 231 were correlated strongly with a different gene set related to cell differentiation and stress responses. These data support the conclusion that CMBs can be used to identify the effects of specific exogenous exposures on normal tissue, and also that these CMBs can be linked to patterns of gene expression.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

TABLE 1
Characteristics of TCGA Pan-Cancer cohort.

| | CMB-RELS | | | |
|---|---|---|---|---|
| Cancer Type | Low | Intermediate | High | Total |
| BLCA | 129 | 128 | 128 | 385 |
| BRCA | 346 | 346 | 346 | 1038 |
| CESC | 87 | 87 | 87 | 261 |
| CHOL | 12 | 12 | 12 | 36 |
| COAD | 138 | 137 | 137 | 412 |
| ESCA | 52 | 52 | 51 | 155 |
| HNSC | 148 | 148 | 148 | 444 |
| KICH | 22 | 22 | 21 | 65 |
| KIRC | 164 | 163 | 163 | 490 |
| KIRP | 89 | 89 | 89 | 267 |
| LGG | 162 | 162 | 162 | 486 |
| LIHC | 121 | 121 | 119 | 361 |
| LUAD | 154 | 154 | 153 | 461 |
| LUSC | 154 | 154 | 153 | 461 |
| OV | 35 | 34 | 34 | 103 |
| PAAD | 60 | 60 | 60 | 180 |
| PRAD | 132 | 132 | 132 | 396 |
| SKCM | 133 | 133 | 133 | 399 |
| STAD | 125 | 124 | 124 | 373 |
| TGCT | 50 | 49 | 49 | 148 |
| THCA | 166 | 166 | 166 | 498 |
| Total | 2479 | 2473 | 2467 | 7419 |

1. A method for training a machine learning model to identify a cellular morphological biomarker (CMB), comprising:
providing a set of histological images to a machine learning model, wherein the set of histological images comprises images of reference tissue and images of test tissue, wherein the test tissue is tissue intentionally exposed to a perturbation, wherein the perturbation comprises at least one chemical compound or at least one source of radiation,
wherein the machine learning model comprises a network layer comprising a set of dictionary elements that represent morphometric features, and the machine learning model identifies a set of CMBs from the images of the reference and test tissues based on the morphometric features.

2. The method of claim 1, further comprising, prior to providing the set of histological images, generating the images of test tissue, wherein generating the images of test tissue comprises:
exposing tissue to the perturbation to generate the test tissue; and
imaging a sample from the test tissue.

3. The method of claim 2, further comprising obtaining a biopsy from the test tissue to generate the sample; and optionally staining the sample with hematoxylin and eosin stain.

4. The method of claim 1, wherein the perturbation is selected from a mutagen, a tumor promoter, a tumor anti-promoter, an anti-inflammatory, and ionizing radiation.

5-7. (canceled)

8. The method of claim 1, wherein the network layer comprises 4 or fewer, 8 or fewer, 16 or fewer, 32 or fewer, 64 or fewer, 128 or fewer, 256 or fewer, 512 or fewer, 1024 or fewer, or 2048 or fewer dictionary elements.

9. (canceled)

10. The method of claim 1, further comprising evaluating the machine learning model to reconstruct a cellular object based on at least one CMB.

11. (canceled)

12. The method of claim 1, wherein the test tissue is murine tissue.

13. The method of claim 12, wherein the murine tissue is genetically modified to induce a phenotype selected from genome instability, membrane perturbation, increased inflammation, tumor susceptibility, and combinations thereof.

14. (canceled)

15. The method of claim 1, wherein the test tissue is selected from a mouse organoid or a human organoid.

16. The method of claim 1, wherein the intentional exposure to the perturbation is in vitro.

17. The method of claim 1, wherein the reference tissue and the test tissue are isogenic to one another.

18. The method of claim 1, wherein the set of histological images comprises images of test tissues which were: (a) exposed to different doses of the perturbation, (b) exposed to the perturbation for different lengths of time, and/or (c) exposed to different numbers of doses.

19-21. (canceled)

22. The method of claim 1, wherein the method further comprises identifying a subset of CMBs, from the set of CMBs, that significantly correlate with the images of the test tissue versus the images of the reference tissue; optionally wherein identifying said subset of significantly correlated CMBs comprises: identifying CMBs with the largest variances in abundance contributing to 95% or more of total data variation, and identifying those that are predictive of test tissue versus reference tissue.

23-24. (canceled)

25. The method of claim 22, wherein the method further comprises generating a CMB perturbation scoring algorithm based on the identified subset of significantly associated CMBs; optionally wherein the CMB perturbation scoring algorithm has the formula:
$$\underline{CMB perturbation score = Intercept + {\sum }_{i = 1}^{N} ( coefficient of {CMB}^{i} ) * ( {CMB}^{i} )}$$
wherein “N” is the number of CMBs in the identified subset of significantly associated CMBs, and the coefficient of CMBⁱ is obtained for each significantly associated CMB using step-wised multivariate linear regression.

26. (canceled)

27. A method of identifying physiological effects of a compound, comprising:
providing one or more histological images of one or more tissue samples from an individual to a machine learning model trained by the method of claim 22,
wherein the trained machine learning model, based on the one or more histological images from the individual, outputs CMB values for the subset of significantly correlated CMBs.

28. The method of claim 27, further comprising calculating a CMB perturbation score for the individual using the CMB perturbation scoring algorithm.

29-34. (canceled)

35. The method of claim 27, wherein the individual is human, and the machine learning model is trained from a set of histological images from a mouse.

36-42. (canceled)

43. A method for generating a reference library for cellular morphological biomarkers (CMBs), comprising:
exposing a collection of murine tissue to a perturbation, wherein the perturbation comprises at least one chemical compound or at least one source of radiation, and each piece of tissue in the collection is acutely exposed or chronically exposed to the perturbation; and
obtaining a histological image of each piece of tissue in the collection and at least one reference piece of murine tissue which has not been exposed to a perturbation.

44. (canceled)

45. The method of claim 43, wherein the murine tissue comprises isogenic mice, inbred mice, heterogeneous mice, and/or genetically modified mice.

46-47. (canceled)

48. The method of claim 43, wherein the perturbation is selected from a mutagen, a tumor promoter, a tumor anti-promoter, an anti-inflammatory, a chemotherapeutic, an immunotherapeutic, and ionizing radiation.

49-52. (canceled)
